(19) United States
(12) Patent Application Publication
(10) Pub. No.: US 2019/0033253 A1
(43) Pub. Date: Jan. 31, 2019

(54) PASSIVE ELECTROCHEMICAL GAS SENSOR BOARD WITH SELF-TEST

(71) Applicant: Honeywell International Inc., Morris Plains, NJ (US)

(72) Inventors: Yangyuan Yu, Shanghai (CN); Liandong Deng, Shanghai (CN); Yong Tang, Shanghai (CN)

(21) Appl. No.: 15/661,491

(22) Filed: Jul. 27, 2017

(51) Int. Cl. G01N 27/416; G01N 33/00; G08B 21/18; G08B 21/14

(52) U.S. Cl. CPC G01N 27/4163; G01N 33/0044; G08B 21/14; G08B 21/182; G01N 33/004

(57) ABSTRACT

A gas detection system. The gas detection system comprises a passive electrochemical (EC) gas sensor, a signal generator electrically coupled to the passive EC gas sensor, a low-pass filter electrically coupled to an output of the passive EC gas sensor, where the low-pass filter has a cut-off frequency below the fundamental frequency of an output of the signal generator, a high-pass filter electrically coupled to the output of the passive EC gas sensor, where the high-pass filter has a cut-off frequency below the fundamental frequency of the signal generator output, a fail indicator that activates when the gas detection system is powered and an amplitude of an output of the high-pass filter is below a first threshold, and a gas detected indicator that activates when the gas detection system is powered and an amplitude of an output of the low-pass filter is above a second configured threshold.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Electrochemical gas sensors (EC gas sensors) measure the concentration of a target gas by oxidizing or reducing the target gas at an electrode, thereby generating an electrical current that can be sensed in an external circuit. Some gases will interact directly with an EC gas sensor that is not provided with an electric voltage bias while other gases will not interact with an EC gas sensor unless it is provided with an appropriate voltage bias to encourage the interactions. EC gas sensors that do not have an electric voltage bias applied across their working electrode (WE) and counter electrode (CE) are referred to as passive EC gas sensors. EC gas sensors that do have an electric voltage bias applied across their WE and CE are referred to as biased EC gas sensors. When the voltage is maintained at a constant level and the current is measured as an indication of a gas concentration, such sensors are referred to as being operated as potentiostatic EC gas sensors. By adapting the bias applied across its WE and CE to maintain a predefined bias voltage, a potentiostatic EC gas sensor may achieve a long service life. By contrast, the working electrode of a passive EC gas sensor can be consumed over time, and the passive EC gas sensor must be replaced more frequently than a potentiostatic EC gas sensor as the WE is used up.

### SUMMARY

In an embodiment, a gas detection system is disclosed. The gas detection system comprises a passive electrochemical (EC) gas sensor, a signal generator electrically coupled to the passive EC gas sensor, a low-pass filter electrically coupled to an output of the passive EC gas sensor, where the low-pass filter has a cut-off frequency below the fundamental frequency of an output of the signal generator, a high-pass filter electrically coupled to the output of the passive EC gas sensor, where the high-pass filter has a cut-off frequency below the fundamental frequency of the output of the signal generator, a passive EC gas sensor fail indicator that activates when the gas detection system is powered and an amplitude of an output of the high-pass filter is below a first threshold, and a gas detected indicator that activates when the gas detection system is powered and an amplitude of an output of the low-pass filter is above a second configured threshold.

In another embodiment, a hazardous gas detector analog board is disclosed. The hazardous gas detector analog board comprises a passive electrochemical (EC) gas sensor configured to pass through a sensor status continuous test signal and to provide an indication of a concentration of a hazardous gas, a sensor status continuous test signal input pin electrically coupled to the passive EC gas sensor, and a high-pass analog filter electrically coupled to an output of the passive EC gas sensor, where the high-pass filter is configured to exhibit a cut-off frequency that is below a fundamental frequency of the sensor status signal, where a signal output of the high-pass filter above a pre-defined threshold provides an indication of operational status of the EC gas sensor.

In yet another embodiment, a method of alerting a concentration of a hazardous gas above a pre-defined alert threshold is disclosed. The method receiving a sensor status continuous test signal by a passive electrochemical (EC) gas sensor of a hazardous gas sensor instrument, passing the sensor status continuous test signal by the passive EC gas sensor to a high-pass filter and to a low-pass filter, providing an indication of a concentration of a hazardous gas by the passive EC gas sensor to the high-pass filter and to the low-pass filter, passing the sensor status continuous test signal by the high-pass filter through to a status signal output, blocking pass through of the indication of the concentration of the hazardous gas by the high-pass filter, passing the indication of the concentration of the hazardous gas by the low-pass filter through to a hazardous gas concentration output, and blocking pass through of the sensor status continuous test signal by the low-pass filter.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a block diagram of a hazardous gas detection instrument according to an embodiment of the disclosure.

FIG. 2 is a block diagram of an analog processing board according to an embodiment of the disclosure.

FIG. 3 is an illustration of an analog processing board according to an embodiment of the disclosure.

FIG. 4 is a flow chart of a method according to an embodiment of the disclosure.

FIG. 5 is a block diagram of a computer system according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

A passive electrochemical (EC) gas sensor circuit board with self-test is taught herein. This circuit board may also be referred to in some contexts as an analog circuit board or an analog board. As the working electrode (WE) of an EC gas sensor is progressively used up, the EC gas sensor reaches a point where it no longer performs its function. When the WE of an EC gas sensor is consumed, the EC gas sensor does not indicate the presence of the gas for which it is designed to detect. When the EC gas sensor is designed to detect hazardous gases, for example to detect CO (carbon monoxide) gas or H2S (hydrogen sulfide) gas, this failure mode of a passive EC gas sensor is dangerous to human beings who may rely on the EC gas sensor to warn them of the gas hazard. The present disclosure teaches a continuous self-test feature of the passive EC gas sensor board that can cause an alert to be presented to the human user when the EC gas sensor is used up.

The passive EC gas sensor board as taught herein receives a test signal that comprises alternating current (AC) frequency content. When the passive EC gas sensor is operable (e.g., when the working electrode is not consumed), the signal frequency content passes through the EC gas sensor to an output of the EC gas sensor. If the EC gas sensor is detecting the presence of a gas, this detection signal is superimposed on the AC frequency content of the test signal, for example added as a direct current (DC) offset to the AC frequency content. Downstream of the EC gas sensor output, a low-pass filter rejects the AC frequency content of the test signal and passes any indication of the concentration of gas through to a gas concentration output. Also downstream of the EC gas sensor output, a high-pass filter rejects DC and AC frequency content below a predefined cut-off frequency and passes the test signal through to a sensor status output. If the WE of the EC gas sensor is used up, the test signal will not pass through the EC gas sensor and the sensor status output will not provide the test signal. Lack of the test signal at the sensor status output can be construed by other circuitry as an EC gas sensor failure condition. The human user can change out a system using the failed EC gas sensor for an alternative system for use in a potentially dangerous work environment, and the failed EC gas sensor can be replaced in the out-of-service system.

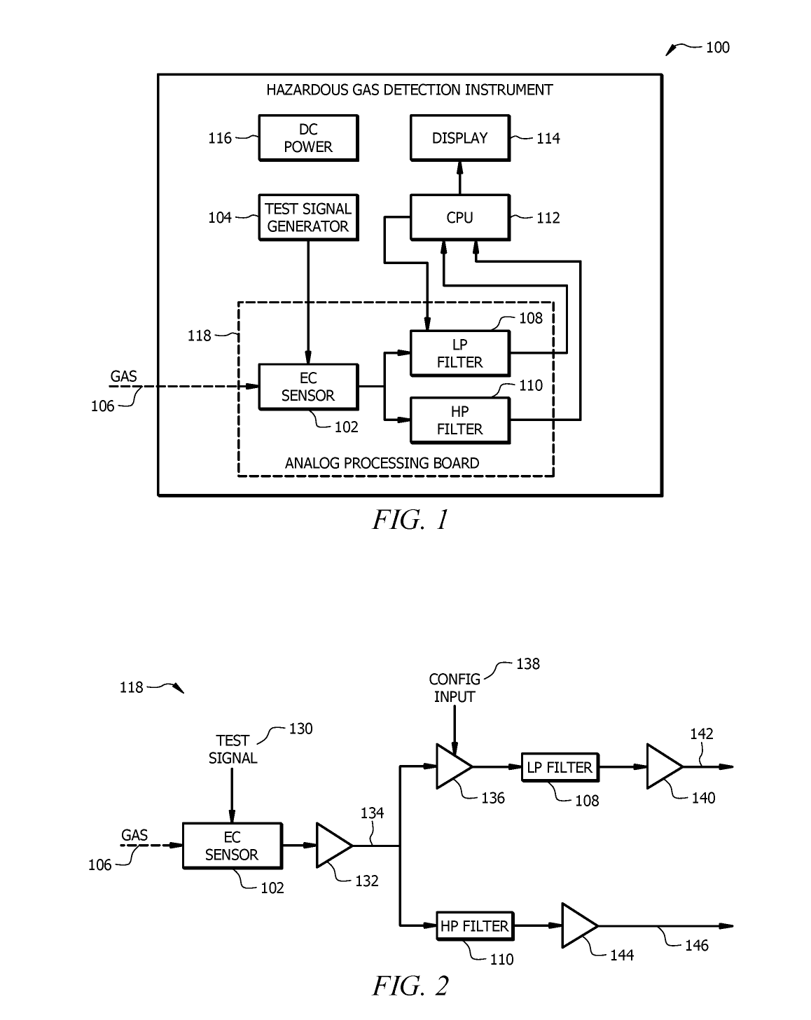

Turning now to FIG. 1, a gas detection system 100 is described. In an embodiment, the system 100 comprises an electrochemical (EC) gas sensor 102, a test signal generator 104, a low-pass (LP) filter (108), a high-pass (HP) filter 110, a processor 112 (which may be referred to as a central processor unit or CPU), a display 114, and a DC power source 116 (e.g., a battery or a DC power supply sourced with AC line power). The EC gas sensor 102, the LP filter 108, and the HP filter 110 may be located on an analog processing board 118. Gas 106 may be diffused into the EC gas sensor 102 if present in the environment of the system 100. The gas detection system 100 may be configured to detect the presence of a hazardous gas, such as CO (carbon monoxide) gas or H2S (hydrogen sulfide) gas. The gas detection system 100 may be used as a portable instrument in a work environment. The gas detection system 100 may be an item of wearable equipment that would be attached to a worker in the work environment. When hazardous gas is present, the display 114 presents an alert or alarm to notify of the hazardous condition. When the EC gas sensor 102 is failed (e.g., when its working electrode is used up), the display 114 provides an alert or alarm to notify of the failure of the EC gas sensor 102. The processor 112 may be implemented as one or more microprocessors, one or more microcontrollers, one or more field programmable gate arrays (FPGAs), one or more application specific integrated circuits (ASICs), one or more complex programmable devices (CPLDs), or a combination thereof.

Turning now to FIG. 2, further details of the analog processing board 118 are described. A test signal 130 may be provided to the EC gas sensor 102, for example output by the test signal generator 104. The test signal 130 may be any signal that comprises an AC frequency content above a cut-off frequency of the HP filter 110. A high-pass filter blocks DC signals and AC signals below the cut-off frequency and passes AC signals having a frequency above the cut-off frequency. It is understood that the cut-off frequency is deemed that point at which amplitude is reduced by 3 decibels (db). In an embodiment, the test signal 130 may be a 3 kilohertz (kHz) sine wave, but in another embodiment, the test signal 130 may comprise a plurality of frequencies. The test signal 130 may have some other wave shape than a sine wave, albeit theoretically a periodic signal may be conceptualized as a series of harmonically related sine waves. The test signal 130 passes through the EC gas sensor 102 and is present on the output of the EC gas sensor 102, provided that the EC gas sensor 102 is operable. When the WE of the EC gas sensor 102 is consumed, for example, the test signal 130 does not pass through the EC gas sensor 102, and its absence can be construed as an indication of failure of the EC gas sensor 102, as described further herein after. The test signal generator 104 that provides the test signal 130 may be an oscillator, a saw-tooth wave generator, a square wave generator, or other component.

When the gas 106 is present to the EC gas sensor 102 (e.g., a sufficient concentration of gas 106 diffuses into the EC gas sensor 102 and contacts the WE), the EC gas sensor 102 establishes a DC signal (a unidirectional electrical current) that is present on the output of the EC gas sensor 102. The amplitude of the DC signal increases as the concentration of the gas 106 increases and decreases as the concentration of the gas 106 decreases. The output of the EC gas sensor 102 can be conceptualized as the superposition of the test signal 130 and any DC signal developed in response to the presence of gas 106. This output may be amplified or otherwise conditioned by a first electrical circuit 132, for example by an amplifier. The output 134 of the first electrical circuit 132 is passed to a second electrical circuit 136, for example an amplifier that is configured by configuration input 138. The processor 112 may provide the configuration input 138. The configuration input may control an amplification gain associated with the second electrical circuit 136. The output 134 of the first electrical circuit 132 is also passed to the HP filter 110.

An output of the second electrical circuit 136 is passed to the LP filter 108. A low-pass filter passes DC signals and AC signals below a cut-off frequency of the low-pass filter. Again, the cut-off frequency is the point where frequency content is attenuated by 3 decibels (db). The cut-off frequency of the LP filter 108 is set at below the fundamental frequency of the test signal 130. For example, the cut-off frequency of the LP filter 108 may be less than 1.2 kHz, less than 600 hertz (Hz), less than 300 Hz, or some other frequency. The LP filter 108 blocks the test signal 130 while passing any signal associated with the presence of gas 106. The output of the LP filter 108 is conditioned by a third electrical circuit 140 to produce a gas sensor signal 142 which is provided to the processor 112. The third electrical circuit 140 may be a voltage follower circuit which limits the maximum voltage of the gas sensor signal 142 whereby to provide safety in a potentially dangerous (e.g., inflammable hydrocarbon gases) work environment. The gas sensor signal 142 provides an indication of the concentration of the gas 106.

The HP filter 110 has a cut-off frequency that is below the fundamental frequency content of the test signal 130 but above the cut-off frequency of the LP filter 108. The cut-off frequency of the HP filter 110 may be above 400 Hz, above 800 Hz, or above 1.4 kHz. The HP filter 110 blocks the component of the output 134 due to the presence of gas 106 which is a DC signal. The component of the output 134 due to the test signal 130, however, passes through the HP filter 110, because the test signal 130 is above the cut-off frequency of the HP filter 110. The output of the HP filter 110 is conditioned by a fourth electrical circuit 144 (e.g., an amplifier) which outputs a detector status signal 146 which is provided to the processor 112.

The LP filter 108 and the HP filter 110 may be implemented with passive circuit elements such as capacitors and resistors. Because of the difference between the test signal 130 and the gas presence signal generated by the EC gas sensor when the gas 106 is present, the quality of the filters 108, 110 need not be high quality (e.g., the per-octave amplitude attenuation of the filter). The electrical circuits 132, 136, 140, 144 may be composed of various operational amplifiers, resistors, and capacitors.

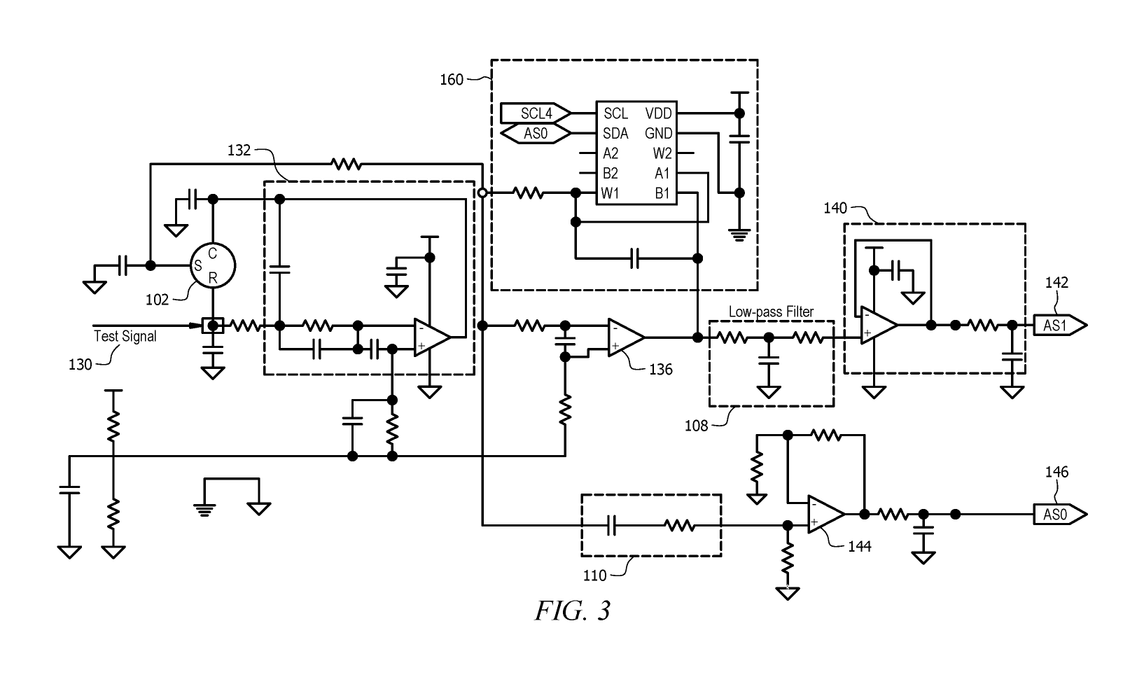

Turning now to FIG. 3, a specific implementation of the analog processing board 118 is discussed briefly. It is understood that the present disclosure is not limited to the specific circuit illustrated in FIG. 3. Other circuits that deviate from that illustrated in FIG. 3 may still take advantage of the novel teachings discussed more broadly above with reference to FIG. 1 and FIG. 2. As shown in FIG. 3, the LP filter 108 may be embodied as a resistor capacitator (RC) low-pass filter (resistor in series with output and capacitor in parallel with output), and the HP filter 110 may be embodied as a RC high-pass filter (resistor and capacitor in series with output). The configuration input 138 may be provided by an electrical circuit 160 that comprises a digital potentiometer that is controlled by the processor 112. The electrical circuit 160 may control a gain of the second electrical circuit 136.

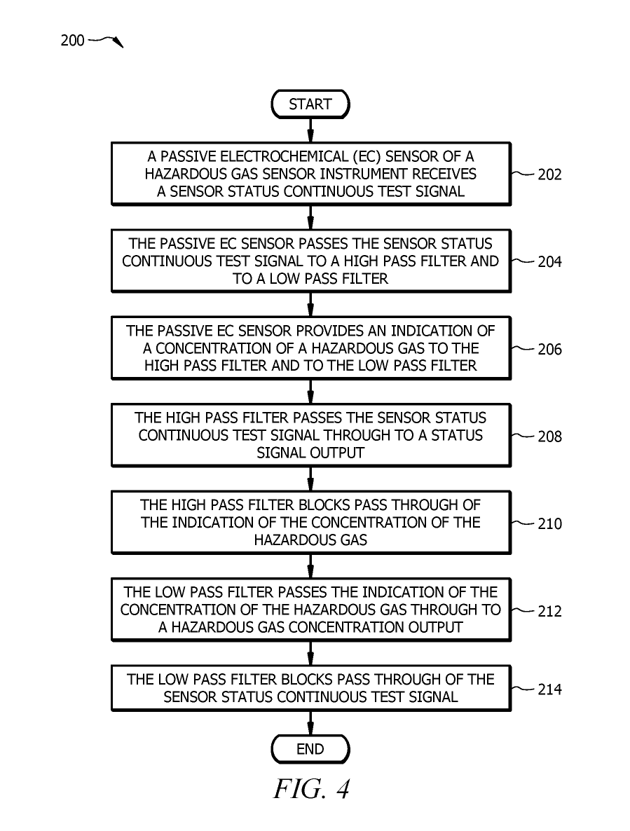

Turning now to FIG. 4, a method 200 is described. At block 202, a passive electrochemical (EC) gas sensor of a hazardous gas sensor instrument receives a sensor status continuous test signal. This test signal may have a periodic frequency content, for example a frequency of at least 1 kHz, at least 2 kHz, at least 3 kHz, or some other frequency. The test signal may be composed of different AC frequencies. At block 204, the passive EC gas sensor passes the sensor status continuous test signal to a high pass filter and to a low pass filter. At block 206, the passive EC gas sensor provides an indication of a concentration of a hazardous gas to the high pass filter and to the low pass filter. The indication of concentration of the hazardous gas may be a DC signal.

At block 208, the high pass filter passes the sensor status continuous test signal through to a status signal output. In an embodiment, the sensor status continuous test signal drops below a second predetermined threshold and causes presentation of a failed EC gas sensor alert. For example, the detector status signal 146 is provided to the processor 112, the processor 112 determines that the detector status signal is less than the second predefined threshold, and the processor 112 commands the display 114 to present a failed EC gas sensor alert. Presentation of the failed EC gas sensor alert may comprise presenting a textual and/or graphic indication on the display 114. Presentation of the failed EC gas sensor alert may further comprise presenting an aural alert such as a bell, a buzzing sound, or other sound. At block 210, the high pass filter blocks pass through of the indication of the concentration of the hazardous gas. Blocking the pass through of the indication of the concentration of the hazardous gas may comprise attenuating an amplitude of the indication by at least 3 decibels.

At block 212, the low pass filter passes the indication of the concentration of the hazardous gas through to a hazardous gas concentration output. In an embodiment, the indication of the concentration of the hazardous gas exceeds a first predefined threshold and causes presentation of a hazardous gas alert. For example, the gas sensor signal 142 is provided to the processor 112, the processor 112 determines that the indication of the concentration of gas exceeds the first predefined threshold, and the processor 112 commands the display 114 to present a hazardous gas alert. Presenting the hazardous gas alert can comprise presenting a textual and/or graphic indication on the display 114. Presentation of the hazardous gas alert may further comprise presenting an aural alert, such as a bell, a buzzing sound, or other sound. When the indication of the concentration of the hazardous gas does not exceed the first predefined threshold, the processor 112 may cause the display 114 to present an indication of the gas concentration, for example a numerical value or some qualitative indication such as “low hazard,” “moderate hazard,” or “high hazard.” At block 214, the low pass filter blocks pass through of the sensor status continuous test signal. Blocking the sensor status continuous test signal may comprise attenuating an amplitude of the continuous test signal by at least 3 decibels.

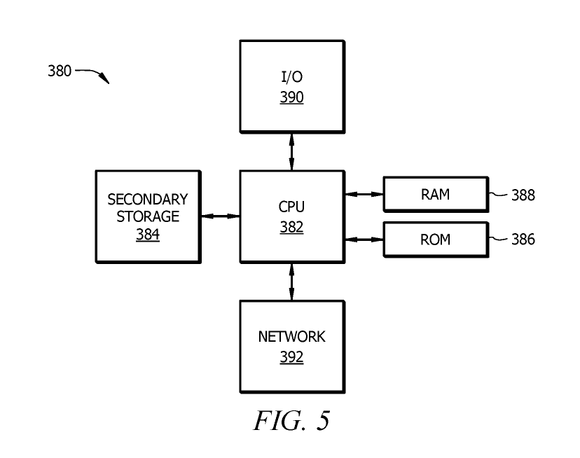

FIG. 5 illustrates a computer system 380 suitable for implementing one or more embodiments disclosed herein. For example, portions of the gas detection system 100 may be considered to be implemented as a computer system. The computer system 380 includes a processor 382 (which may be referred to as a central processor unit or CPU) that is in communication with memory devices including secondary storage 384, read only memory (ROM) 386, random access memory (RAM) 388, input/output (I/O) devices 390, and network connectivity devices 392. The processor 382 may be implemented as one or more CPU chips.

It is understood that by programming and/or loading executable instructions onto the computer system 380, at least one of the CPU 382, the RAM 388, and the ROM 386 are changed, transforming the computer system 380 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the computer system 380 is turned on or booted, the CPU 382 may execute a computer program or application. For example, the CPU 382 may execute software or firmware stored in the ROM 386 or stored in the RAM 388. In some cases, on boot and/or when the application is initiated, the CPU 382 may copy the application or portions of the application from the secondary storage 384 to the RAM 388 or to memory space within the CPU 382 itself, and the CPU 382 may then execute instructions that the application is comprised of In some cases, the CPU 382 may copy the application or portions of the application from memory accessed via the network connectivity devices 392 or via the I/O devices 390 to the RAM 388 or to memory space within the CPU 382, and the CPU 382 may then execute instructions that the application is comprised of. During execution, an application may load instructions into the CPU 382, for example load some of the instructions of the application into a cache of the CPU 382. In some contexts, an application that is executed may be said to configure the CPU 382 to do something, e.g., to configure the CPU 382 to perform the function or functions promoted by the subject application. When the CPU 382 is configured in this way by the application, the CPU 382 becomes a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identification (RFID), and/or other air interface protocol radio transceiver cards, and other well-known network devices. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from a hard disk, floppy disk, optical disk (these various disk based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 380 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 380 to provide the functionality of a number of servers that are not directly bound to the number of computers in the computer system 380. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 380, at least portions of the contents of the computer program product to the secondary storage 384, to the ROM 386, to the RAM 388, and/or to other non-volatile memory and volatile memory of the computer system 380. The processor 382 may process the executable instructions and/or data structures in part by directly accessing the computer program product, for example by reading from a CD-ROM disk inserted into a disk drive peripheral of the computer system 380. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 380.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 380 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A gas detection system, comprising:
a passive electrochemical (EC) gas sensor;
a signal generator electrically coupled to the passive electrochemical gas sensor;
a low-pass filter electrically coupled to an output of the passive electrochemical gas sensor, where the low-pass filter has a cut-off frequency below the fundamental frequency of an output of the signal generator;
a high-pass filter electrically coupled to the output of the passive electrochemical gas sensor, where the high-pass filter has a cut-off frequency below the fundamental frequency of the output of the signal generator;
a passive electrochemical gas sensor fail indicator that activates when the gas detection system is powered and an amplitude of an output of the high-pass filter is below a first threshold; and
a gas detected indicator that activates when the gas detection system is powered and an amplitude of an output of the low-pass filter is above a second configured threshold.

2. The gas detection system of claim 1, where the electrochemical gas sensor is operable to detect the presence of carbon monoxide (CO).

3. The gas detection system of claim 1, where the electrochemical gas sensor is operable to detect the presence of hydrogen sulfide (H2S) gas.

4. The gas detection system of claim 1, further comprising a processor that is electrically coupled to the output of the high-pass filter and the output of the low-pass filter, compares the amplitude of the output of the high-pass filter to the first threshold, compares the amplitude of the output of the low-pass filter to the second threshold, controls the activation of the electrochemical gas sensor fail indicator based on comparing the amplitude of the output of the high-pass filter to the first threshold, and controls the activation of the gas detected indicator based on comparing the amplitude of the output of the low-pass filter to the second threshold.

5. The gas detection system of claim 4, further comprising a first amplifier and a digital potentiometer electrically coupled to the first amplifier, where a gain of the first amplifier is controlled by the digital potentiometer and the digital potentiometer is controlled by the processor, where the output of the passive electrochemical gas sensor is electrically coupled to the input of the first amplifier, and where an output of the first amplifier is electrically coupled to the input of the low-pass filter.

6. The gas detection system of claim 5, further comprising a voltage follower amplifier circuit, where the output of the low-pass filter is electrically coupled to an input of the voltage follower amplifier, and where an output of the voltage follower amplifier circuit is electrically coupled to the processor.

7. The gas detection system of claim 4, further comprising a second amplifier, where the output of the high-pass filter is electrically coupled to the input of the second amplifier, and an output of the second amplifier is electrically coupled to the processor.

8. A hazardous gas detector analog board, comprising:
a passive electrochemical (EC) gas sensor configured to pass through a sensor status continuous test signal and to provide an indication of a concentration of a hazardous gas;
a sensor status continuous test signal input pin electrically coupled to the passive electrochemical gas sensor; and
a high-pass analog filter electrically coupled to an output of the passive electrochemical gas sensor, where the high-pass filter is configured to exhibit a cut-off frequency that is below a fundamental frequency of the sensor status signal, where a signal output of the high-pass filter above a pre-defined threshold provides an indication of operational status of the electrochemical gas sensor.

9. The hazardous gas detector analog board of claim 8, where the high-pass analog filter comprises a capacitor and a resistor in series with the output of the passive electrochemical gas sensor and the output of the high-pass filter.

10. The hazardous gas detector analog board of claim 9, where the cut-off frequency of the high-pass filter is above 800 Hz.

11. The hazardous gas detector analog board of claim 8, further comprising a low-pass filter electrically coupled to the output of the passive electrochemical gas sensor, where the low-pass filter is configured to exhibit a cut-off frequency that is below the cut-off frequency of the high-pass filter and operable to output the indication of the concentration of hazardous gas.

12. The hazardous gas detector analog board of claim 11, where the cut-off frequency of the low-pass filter is less than 600 Hz.

13. The hazardous gas detector analog board of claim 11, further comprising a voltage follower circuit electrically coupled to an output of the low-pass filter and operable to output the indication of the concentration of hazardous gas.

14. The hazardous gas detector analog board of claim 8, wherein the passive electrochemical gas sensor is configured to provide an indication of the concentration of CO (carbon monoxide) gas or of H2S (hydrogen sulfide) gas.

15. A method of alerting a concentration of a hazardous gas above a pre-defined alert threshold, comprising:
receiving a sensor status continuous test signal by a passive electrochemical (EC) gas sensor of a hazardous gas sensor instrument;
passing the sensor status continuous test signal by the passive electrochemical gas sensor to a high-pass filter and to a low-pass filter;
providing an indication of a concentration of a hazardous gas by the passive electrochemical gas sensor to the high-pass filter and to the low-pass filter;
passing the sensor status continuous test signal by the high-pass filter through to a status signal output;
blocking pass through of the indication of the concentration of the hazardous gas by the high-pass filter;
passing the indication of the concentration of the hazardous gas by the low-pass filter through to a hazardous gas concentration output; and
blocking pass through of the sensor status continuous test signal by the low-pass filter.

16. The method of claim 15, wherein blocking pass through of the indication of the concentration of the hazardous gas by the high-pass filter comprises attenuating an amplitude of the indication of the concentration by at least 3 decibels.

17. The method of claim 15, wherein blocking pass through of the sensor status continuous test signal by the low-pass filter comprises attenuating an amplitude of the sensor status continuous test signal by at least 3 decibels.

18. The method of claim 15, wherein hazardous gas is CO (carbon monoxide) gas or H2S (hydrogen sulfide) gas.

19. The method of claim 15, further comprising presenting a hazardous gas alert based on the hazardous gas concentration output exceeding a first predefined threshold.

20. The method of claim 15, further comprising presenting a failed EC sensor alert based on the status signal output dropping below a second predefined threshold.
